(19) United States
(12) Patent Application Publication
(10) Pub. No.: US 2026/0365962 A1
(43) Pub. Date: Nov. 19, 2026

(54) METHOD OF MONITORING AND CONTROLLING THE FOAM WITHIN A VESSEL

(71) Applicant: Genentech, Inc., South San Francisco, CA (US)

(72) Inventors: Edward Hon Man CHAN, San Francisco, CA (US); Neria DANIEL, Foster City, CA (US); Carmen GUAN, San Francisco, CA (US); Christian James RANDECKER, Alameda, CA (US); Hongbo Mike SU, Millbrae, CA (US)

(73) Assignee: Genentech, Inc., South San Francisco, CA (US)

(21) Appl. No.: 19/181,573

(22) Filed: Apr. 17, 2025

Related U.S. Application Data
(60) Provisional application No. 63/636,263, filed on Apr. 19, 2024.

(51) Int. Cl. C12M 1/21; C12M 1/34; C12M 1/36

(52) U.S. Cl. CPC C12M 41/02; C12M 41/32; C12M 41/44; C12M 41/48

(57) ABSTRACT

The application relates to a method of monitoring and controlling the foam within a vessel comprising a liquid. The method comprises measuring the thickness of the foam within a vessel comprising a liquid and controlling the dosage of an antifoaming additive to be added to the liquid based on the measured thickness of the foam. The thickness of the foam is determined based on the difference between the level of a phase surface and the level of the liquid. The level of the phase surface is determined by a signal emitted towards and reflected from the phase surface. An apparatus for conducting the method, and a further method of producing biologics from cells is also disclosed.

### CROSS REFERENCE TO RELATED APPLICATION

This application claims priority to U.S. Provisional Application No. 63/636,263, filed Apr. 19, 2024, which is incorporated by reference in its entirety.

### TECHNICAL FIELD

This invention relates to a method of monitoring and controlling the foam within a vessel comprising a liquid.

### BACKGROUND

Foam formation in bioreactors during manufacturing processes is a known problem. Foaming is undesirable due to adverse effects on the manufacturing process. Examples of adverse effects include entrapment of cells in the foam, pressure surge due to clogged vent filters, risk of overflow, and contamination. There is therefore a need to reduce foam formation within bioreactors.

However, this is tempered by the desire to improve manufacturing output, which may require conditions that are favorable to foam formation (for example bubbling gas through the liquid in a bioreactor vessel, agitation, or a vigorous culture).

As such, it can be undesirable or impossible to completely eliminate foam formation in bioreactors for a given manufacturing process. One way to control foam formation is to add anti-foam additives to the liquid in the bioreactor vessel. The addition of anti-foam additives is not a complete solution as too much anti-foam additive can impact negatively on production yield and quality. Attempts have therefore been made to hold the foam formed within a bioreactor within a tolerable volume of foam.

This has been achieved by the periodic addition of anti-foam additives to the bioreactor during manufacturing processes. When the presence of excess foam is identified, anti-foam additive is introduced to return the amount of foam to within the tolerable range. In order to achieve this reduction of foam to a tolerable range it has been necessary to monitor the amount of foam within a bioreactor.

Monitoring the volume of foam within a bioreactor is problematic for a number of reasons. Bioreactors may be formed of opaque materials, such as stainless steel, therefore preventing direct observation of the foam in the vessel. Foams may also accumulate around a vessel wall more than remote from vessel walls, therefore, even if the bioreactor was formed of a transparent material, any visual reading taken may be a false representation of the amount of foam in the vessel. Such methods further require personnel to take and act upon such a reading.

Efforts have been made to automate monitoring of the foam thickness within bioreactors and take indirect or non-visual readings, such as US2021/355427A1 which discloses the use of vision systems in biomanufacturing processes. Solutions have included use of cameras located within the vessel or arranged to view the contents through a viewing window, however cameras can suffer from obstruction, the output requires manual review by personnel. Another effort outlined by Endress+Hauser utilizes a reduction in radar signal strength to detect foam appearance; however this does not provide a quantitative foam volume measurement required for guiding a precise antifoam addition in a bioreactor. Capacitance sensors have also been deployed in the form of adherent patches attached to the side walls of the bioreactor vessel, e.g., a single-use bag. However, such patches only provide a reading of the foam thickness adjacent to the vessel wall in the vicinity of the patches. This provides information that may not be representative of the entire liquid surface and therefore may produce erroneous or unreliable readings. Furthermore, where the bioreactor vessel is formed of a single-use bioreactor or bag it is often necessary to dispose of the capacitance sensor following the manufacturing process which increases waste and expense.

Previous solutions also do not address the issue that the liquid level within the bioreactor vessel may change as a result of normal manufacturing processes, for example liquid volume may change due to the introduction of reagents (such as fresh medium or supplements). This further complicates determination of the thickness of the foam on a liquid within a bioreactor.

The present invention seeks to at least partially address the above identified problems.

### BRIEF SUMMARY OF THE DISCLOSURE

In accordance with the present invention there is provided a method of monitoring and controlling foam within a vessel comprising a liquid, the method comprising:
a) measuring the thickness of the foam within a vessel comprising a liquid; and
b) controlling the dosage of an antifoaming additive to be added to the liquid based on the measured thickness of the foam;
wherein the thickness of the foam in step a is determined based on the difference between the level of a phase surface and the level of the liquid; and
wherein the level of the phase surface is determined by a signal emitted towards and reflected from the phase surface.

A “phase surface” is the interface between two states of matter. For example, in the present case this may be the surface between the foam and gas in the head space of the vessel, or (if no foam is present) this may be the surface between the liquid and the gas in the head space of the vessel. The level of the phase surface or the phase surface level means, for example, height of the phase surface cumulative with the liquid height in a vessel. The phase surface may be distinct or continuous (e.g. the transition between foam state and the gas state is clearly defined in the case of distinct, or the transition between foam state and the gas state is indistinct with a transition region between the two states). The level of the liquid is determined independently of the thickness of the foam. The level of a liquid or the liquid level means, for example, height of the liquid in a vessel. The level of the liquid may be determined from the quantity of liquid present in the vessel in the absence of the foam i.e. prior to foam formation, and/or may be determined using a method that is largely independent of the presence or absence of the foam. For example, the level of the liquid may be determined by measurement of the mass of the content in a vessel as liquid (even though the mass includes the mass of the liquid and the mass of the foam, the mass of the foam is typically not significant compared to the mass of the liquid, and so the mass may be assumed to correlate with liquid level). “Quantity” here may be based on any appropriate property that may be used to determine the level of the liquid within the vessel, such as mass, volume, depth, and from each of which a “level of the liquid” may be determined. “Thickness of the foam” may be a relative measure, in comparison to the level of the liquid, or an absolute measure, e.g. the distance between the phase surface and a reflected signal receiver, for example. Thickness of the foam or the foam thickness means, for example, the difference between the level of a phase surface and the level of the liquid.

The level of the phase surface may be determined based on a time elapsed between emission of the signal and detection of the reflected signal. The level of the phase surface may therefore be determined based on the time of flight of an interrogating signal to be sent, and reflected from, the phase surface.

The signal may comprise electromagnetic radio waves, or radio waves. The signal may be any signal suitable to provide the intended function, wherein the signal permits the recording of time of flight in order to determine the phase surface level. It will be apparent that some types of signals are appropriate for a given medium, while others are less appropriate for a given medium, e.g. the medium is transparent to, or an absorber of, that particular signal. The medium should be a reflector of the chosen signal.

The signal may comprise radio waves in the range 26 GHz-170 GHz. Radio signals in the aforementioned range are resistant to degradation/attenuation from water droplets suspended in the head space (e.g. from sparging).

The signal may be emitted towards and reflected from varying locations of the phase surface. In this manner the level of the phase surface may be determined as an average, or a “high spot” (i.e., area of phase surface that is arranged closer to the receiver or is at a greater distance from the liquid level relative to other areas of the phase surface). This accounts for various phenomena, such as non-uniform foam formation, foam aggregation, clumping, gathering, and/or the presence of ameniscus.The acceptable thickness of the foam may vary across a phase surface, so a larger amount of foam may be permitted in regions of the phase surface remote from, e.g., filters or other elements of the vessel that are negatively impacted by excess foam.

The level of the liquid may be determined by volume determination and/or determination of a dimension associated with the liquid. The volume may be predetermined (i.e., a known volume of liquid is added to the vessel of a given geometry), or directly determined via, e.g. liquid level sensor, capacitance sensor, guided wave radar and the like. Alternatively, the volume of liquid may be indirectly determined, such as via measuring the mass of the fluid in the vessel and then converting to the liquid volume via density calculations and vessel geometry. The method used to determine liquid volume may be varied during use of the vessel with some methods being more appropriate at differing stages, e.g., predetermined at the start of use of the vessel, with direct and/or indirect determination used at later time point(s).

The level of the liquid may be determined by volume determination associated with the liquid, and wherein the volume determination is based on a mass of the liquid.

The level of the phase surface and the level of the liquid may each be determined substantially simultaneously. In this way the thickness of the foam may be monitored in real time.

The dosage of an antifoaming additive may be controlled based on the measured thickness of the foam. Especially, it may be controlled by the comparison of the measured thickness of the foam to a threshold thickness of the foam. When the amount of foam surpasses the threshold thickness of the foam, the dosage of the antifoaming additive may be altered in order to ensure the amount of foam remains below the threshold. “Dosage” in this application may refer to any suitable measure or regimen for applying the antifoaming agent to the liquid in order to maintain the thickness of the foam within desired limits. Dosage may therefore be varied by changing time intervals between additions of a constant quantity, or the quantity and/or concentration of the antifoaming additive in an addition may be changed and the time between dosage fixed, or a combination of the aforesaid. For example, the dosage may comprise no addition of the antifoaming additive when the measured thickness of the foam is below the threshold thickness of the foam, and addition of an aliquot (e.g. a constant quantity) of the antifoaming additive when the measured thickness of the foam is above the threshold thickness of the foam.

The dosage of the antifoaming additive may, at least in part, be determined by the deviation of the determined level from the threshold level. In this way the dosage may be altered according to the amount of foam within the vessel, thereby minimizing needless or excess addition of antifoaming agent.

The threshold level may, at least in part, be determined based upon an available head space in the vessel. The vessel comprising liquid typically further includes a head space located above the liquid level and normally occupied by a gas. The head space may vary according to the variation in the liquid level and the constant or maximum volume of the vessel. Accordingly, the acceptable thickness of the foam may vary depending on the available head space within the vessel at any given moment. A relatively higher liquid level may result in a lower amount of head space, and in turn, a lower acceptable thickness of the foam. The reverse is also true, a relatively lower liquid level may result in a relatively larger amount of head space, thereby increasing the acceptable thickness of the foam within the vessel. Accordingly, the threshold may account for the head space within the vessel and changes in the head space. The threshold may vary according to the acceptable thickness of the foam within the vessel at any given moment.

The threshold level may, at least in part, be set at a specified level of the foam in the vessel relative to the level of the liquid. The threshold level may, at least in part, be set at a rate of change of the thickness of the foam, with antifoaming agent added when a rapid increase of the thickness of the foam is detected within the vessel.

The dosage of the antifoaming additive may be controlled by adjusting the interval of adding the antifoaming additive and/or the amount of the antifoaming additive added in one instance. As discussed elsewhere, dosage may be varied by changing time intervals between additions of a constant quantity, or the quantity and/or concentration of an addition may be changed and the time between dosage fixed, or a combination of the aforesaid. Here “instance” means a specific, individual, dose that is administered to the vessel. The instant dose may therefore be unique in the quantity of antifoaming agent provided relative to other instant doses which have previously, or will subsequently, be administered to the vessel. The total dosage of the antifoam shall also consider the impact of the antifoam additives to the cell culture performance. The higher negatively impacted cell lines by the antifoam additives shall be more prudent for the amount of antifoam allowed.

The vessel may be a bioreactor. The vessel may be a bioreactor of a volume in the range of from about 1 L to about 50,000 L; however, as the skilled person will appreciate, the present method may be adapted for a bioreactor of any given size, shape, and dimensions.

The liquid may be a cell culture fluid comprising cell culture medium and cells. Advantageously, the present method may be adapted (e.g. on the fly) to accommodate changing liquid properties, such as mass, density, propensity to form foam, degree of foam formation, and foam stability, all of which may change over the typical time-course of a cell culture process.

The method may be performed for a duration of a batch culture, a fed-batch culture, or a perfusion culture process.

The method may be repeated multiple times for the duration of the culture. Advantageously, the method may be performed during the entire course of the culture, if desired, thereby ensuring the thickness of the foam is controlled within the tolerable level automatically.

The repetition may be prompted by addition or subtraction of an amount of liquid from the vessel and/or the elapsing of a period of time. The method may be therefore performed, “on demand”, either as and when, on a whim, or at times where a rapid change in the thickness of the foam is expected (e.g. on the addition of an agent with known foam forming properties).

The method may be repeated substantially continuously for the duration of the culture. A culture may have any suitable duration for the culture to complete or reach a desired end state. This duration may be minutes, hours, days, weeks, or months in length. As an example, a signal with a refresh rate of the order of 5Hz may be used to provide 5 readings per second. Such a rate is suitably short as to capture rapid changes in the foam thickness. Therefore “continuously” in the context of this application means repeated interrogations of the foam thickness rather than a non-stop single interrogation of the foam thickness, which would provide an average foam thickness over a time course and obfuscate peaks in the foam thickness.

In accordance with a further embodiment there is provided a method of producing a biologic from cells, comprising:
a) culturing cells in a cell culture fluid in a bioreactor;
b) measuring the thickness of a foam within the bioreactor comprising the cell culture fluid;
c) controlling the dosage of an antifoaming additive to be added to the cell culture fluid based on the measured thickness of the foam; and
d) obtaining the biologic produced by the cultured cells;
wherein the thickness of the foam in step b is determined based on the difference between the level of a phase surface and the level of the cell culture fluid; and
wherein the phase surface is determined by a signal emitted towards and reflected from the phase surface.

The level of the phase surface may be determined based on a time elapsed between emission of the signal and detection of the reflected signal.

The signal may comprise electromagnetic radio waves.

The signal may comprise radio waves in the range 26 GHz-170 GHz., The signal may optionally comprise 80 GHz radio waves. Radio signals in the aforementioned range are considered to be a compromise between a need for resistant to degradation/attenuation from water droplets suspended in the head space (e.g. from sparging) and a need to reduce the penetration through the foam which is inherent for a higher frequency radar signal.

The signal may be emitted towards and reflected from varying locations of the phase surface.

The level of the cell culture fluid may be determined by volume determination and/or determination of a dimension associated with the cell culture fluid.

The level of the cell culture fluid may be determined by volume determination associated with the cell culture fluid, and wherein the volume determination is based on a mass of the cell culture fluid.

The level of the phase surface and the level of the cell culture fluid may each be determined substantially simultaneously.

The dosage of an antifoaming additive may be controlled by the comparison of the measured thickness of the foam to a threshold thickness of the foam.

The dosage of the antifoaming additive may, at least in part, be determined by the deviation of the determined level from the threshold level.

The threshold level may, at least in part, be determined based upon an available head space in the vessel.

The dosage of the antifoaming additive may be controlled by adjusting the interval of adding the antifoaming additive and/or the amount of the antifoaming additive added in one time.

The bioreactor may have any suitable volume. For example, the bioreactor may have a volume in the range of from about 1 L to about 50,000 L.

The cell culture fluid may comprise cell culture medium and cells.

Steps b) and c) may be performed for a duration of a batch culture, a fed-batch culture, or a perfusion culture.

The steps b) and c) may be repeated multiple times for the duration of the culture.

The repetition may be prompted by addition or subtraction of an amount of cell culture fluid from the vessel and/or the elapsing of a period of time.

The steps b) and c) may be repeated substantially continuously for the duration of the culture.

Step d) may be performed only at about the end of the duration of the culture.

Step d) may comprise isolating the biologic from the cell culture fluid.

The biologic may be at least one recombinant polypeptide and the cells may be or comprise eukaryotic cells comprising a polynucleotide encoding the recombinant polypeptide.

The at least one recombinant polypeptide may be selected from a fusion protein, an antibody, an antigen, an enzyme, or a vaccine.

In accordance with a still further embodiment there is provided an apparatus for controlling the thickness of the foam within a vessel comprising a liquid, the apparatus comprising:
a vessel configured to comprise a liquid;
phase surface level determining means, configured to determine the level of the phase surface, which comprises;
an emitter, configured to emit a signal towards a phase surface;
a receiver, configured to detect a reflected signal from the phase surface;
liquid level determining means, configured to determine the level of the liquid in the vessel; and
a processor, configured to;
determine the thickness of a foam based on the difference between the level of the phase surface and the level of the liquid; and
instruct the dosage of an antifoaming additive to be added to the liquid based on the determined thickness of the foam.

Here, “vessel configured to comprise a liquid” means a vessel capable of containing and reserving a volume of liquid such that the vessel may be used to complete a relevant task, e.g. cell culture and/or biologic production.

The processor is typically arranged in communication with the emitter, receiver, liquid level determining means such that outputs from each may be received by the processor for determining the thickness of a foam based on the difference between the level of the phase surface and the level of the liquid. The processor may be further arranged in communication with an output such that the processor is able to issue the instructions regarding the dosage of antifoaming additive. The output may be any suitable so that changes in dosage of the antifoaming agent are affected.

The phase surface level determining means may determine the level of the phase surface based on a time elapsed between emission of the signal toward the phase surface and detection of the reflected signal. Accordingly, the apparatus may be capable of logging the time a signal is emitted and then received at the emitter and receiver respectively, such as either locally at the respective emitter and receiver, and then such times are passed to the processor, or by the processor instructing emission and awaiting detection via the emitter and receiver.

The emitter and/or receiver may be arranged substantially perpendicular to the phase surface of the liquid in the vessel. In this way the reflected signal is maximized.

The emitter and/or receiver may be arranged above the top of the vessel.

The emitter and/or receiver may be arranged on an outside of the vessel to respectively emit or detect signal to an inside of the vessel. In the further alternative, the emitter and/or receiver may at least partially pass through a wall of the vessel. Locating the emitter and/or receiver outside the vessel is advantageous in facilitating ease of inspection, testing, and maintenance if the receiver and/or emitter. The most important advantage for the radar mounting outside of the bioreactor is that it is the outside of sterile boundary of a bioreactor so no cleaning and sterilization are needed for the next use.

The emitter and/or receiver may be arranged on an inside of the vessel to respectively emit or detect signal within the vessel. Locating the emitter and/or receiver inside the vessel is advantageous in that the method may be used with vessels formed of materials that are opaque to the emitted signal, as the signal need not traverse the opaque vessel and instead may be detected by the internal receiver.

The emitter may be configured to emit any of: electromagnetic radio waves; or radio waves.

The emitter may be configured to emit radio waves in the range 26 GHz-170 GHz.

The liquid level determining means may be selected from a load cell, a scales, a ranging apparatus, and a guided wave radar. As discussed above, the liquid level determining means may determine the liquid level directly (in the case of ranging apparatus or guided wave radar) or indirectly via density and/or volume calculations in the case of a load cell or weighing scales. The liquid level determining means may include means to determine the mass of the liquid (such as, but not limited to, the load cell, or scales), thereby allowing the volume to be calculated using the density of the liquid and vessel geometry.

The vessel may be a bioreactor. The bioreactor may have a volume in the range of from about 1 L to about 50,000 L.

The vessel may formed, at least in part, of a signal-transparent material, and wherein the emitter and/or receiver is arranged to respectively emit or detect a signal through the signal-transparent material. Using a material that is transparent to the signal is advantageous in that the emitter and/or receiver may be arranged outside the vessel, thereby isolating the emitter and/or receiver from the contents of the vessel.

The top of the vessel may be formed of a signal-transparent or signal-transparent material. The vessel may be advantageously formed of a material that is translucent or transparent to the signal, an appropriate material may be selected depending on whether the signal comprises electromagnetic radio waves; or radio waves such that the material is translucent/transparent to the signal. Translucent/transparent here means that the signal is attenuated and/or degraded little or not at all on passing through the material.

The vessel may be formed, at least in part, of a radio-opaque material. The vessel may be provided with a window formed of a signal transparent or translucent material with the rest of the vessel formed of a signal opaque material. This may be for performance, economic, structural, or other reasons where the benefits conferred by the signal opaque material warrant its use rather than forming the entire vessel from a signal transparent or translucent material.

The vessel may be formed, at least in part, of stainless steel; glass; ceramic; and/or a plastic film.

The vessel may comprise at least a partial stainless steel shell and a plastic film liner.

The processor may control the dosage of an antifoaming additive by comparing the determined thickness of the foam to a threshold thickness of the foam.

The processor controls the dosage of an antifoaming additive by determining the deviation of the determined level from the threshold level.

The processor may be configured to determine the thickness of the foam and instruct the dosage of an antifoaming additive repeatedly and/or continuously during a cell culture duration. For example, the processor may be configured to instruct the dosage of an aliquot (e.g. a constant quantity) of the antifoaming additive when the measured thickness of the foam is above the threshold thickness of the foam.

The apparatus may further comprises an antifoaming additive injector configured to automatically inject the antifoaming additive into the vessel when the processor instructs the addition of the antifoaming additive. The antifoaming additive injector is arranged in communication with the processor such that the antifoaming additive injector acts on instruction from the processor.

### BRIEF INTRODUCTION OF THE DRAWINGS

An embodiment of the invention is further described hereinafter, by way of example, with reference to the accompanying drawings, in which:

FIG. 1 shows a cross section of a vessel according to a first embodiment;

FIG. 2 shows a second cross section of a vessel according to the first embodiment;

FIG. 3 shows a third cross section of a vessel according to the first embodiment;

FIG. 4 shows a cross section of a vessel according to a second embodiment;

FIG. 5 is a plot showing the change in the phase surface level, the liquid level, and load cell output plotted with time;

FIG. 6 is a plot showing the measured foam thickness with batch feeds plotted with time; and

FIG. 7 is a plot showing the measured foam thickness, various thresholds, and antifoam addition timing with time.

FIG. 8 is a diagram of an example cell culture system comprising an automatic antifoam monitoring and addition system.

FIG. 9 is a diagram of a cell culture run that depicts foam thickness (black line) in inches (Y-axis) in response to various threshold foam thickness settings (gray dashed line) during cell culture and in response to additions of antifoam when the antifoam addition pump is on (vertical dotted gray lines at X-axis) at various time points (X axis), and the weight of the contents of the cell culture vessel as cell culture progresses (black dashed line).

### DETAILED DESCRIPTION

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

For the avoidance of doubt, it is hereby stated that the information disclosed earlier in this specification under the heading “Background” is relevant to the invention and is to be read as part of the disclosure of the invention.

All publications, patent applications, patents, and other references mentioned herein are incorporated by reference in their entirety. In case of conflict, the present specification, including definitions, will control.

### Definitions

The terms used in this specification generally have their ordinary meanings in the art, within the context of this disclosure and in the specific context where each term is used.

Certain terms are discussed below, or elsewhere in the specification, to provide additional guidance to the practitioner in describing the compositions and methods of the present disclosure and how to make and use them.

As used herein, the use of the word “a” or “an” when used in conjunction with the term “comprising” in the claims and/or the specification can mean “one,” but it is also consistent with the meaning of “one or more,” “at least one” and “one or more than one.” The terms “comprise(s),” “include(s),” “having,” “has,” “can,” “contain(s)” and variants thereof, as used herein, are intended to be open-ended transitional phrases, terms or words that do not preclude the possibility of additional acts or structures. The present disclosure also contemplates other embodiments “comprising,” “consisting of” and “consisting essentially of,” the embodiments or elements presented herein, whether explicitly set forth or not.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The term “cell culture fluid” refers to a fluid that may be used for culturing cells, such as (an aqueous) fluid comprising a “cell culture medium” or “culture medium”. The terms “cell culture medium” and “culture medium” refer to a nutrient solution used for growing mammalian cells that typically provides at least one component from one or more of the following categories:
1) an energy source, usually in the form of a carbohydrate such as glucose;
2) all essential amino acids, and usually the basic set of twenty amino acids plus cysteine;
3) vitamins and/or other organic compounds required at low concentrations;
4) free fatty acids; and
5) trace elements, where trace elements are defined as inorganic compounds or naturally occurring elements that are typically required at very low concentrations, usually in the micromolar range.

The nutrient solution can optionally be supplemented with one or more components from any of the following categories:
1) hormones and other growth factors as, for example, insulin, transferrin, and epidermal growth factor;
2) salts and buffers as, for example, calcium, magnesium, and phosphate;
3) nucleosides and bases such as, for example, adenosine, thymidine, and hypoxanthine; and
4) protein and tissue hydrolysates.

“Culturing” a cell refers to contacting a cell with a cell culture medium under conditions suitable to the survival and/or growth and/or proliferation of the cell.

“Batch culture” refers to a culture in which all components for cell culturing (including the cells and all culture nutrients) are supplied to the culturing bioreactor at the start of the culturing process.

“Fed-batch cell culture,” as used herein refers to a batch culture wherein the cells and culture medium are supplied to the culturing bioreactor initially, and additional culture nutrients are fed, continuously or in discrete increments, to the culture during the culturing process, with or without periodic cell and/or product harvest before termination of culture.

“Perfusion culture,” sometimes referred to as continuous culture, is a culture by which the cells are restrained in the culture by, e.g., filtration, encapsulation, anchoring to microcarriers, etc., and the culture medium is continuously, step-wise or intermittently introduced (or any combination of these) and removed from the culturing bioreactor.

As used herein, the term “cells” refers to animal cells (e.g. mammalian cells), bacteria cells (e.g. E. Coli cells), fungal cells (e.g. yeast cells), cultured cells, host cells, recombinant cells and recombinant host cells. Such cells are generally cell lines obtained or derived from mammalian tissues or fungi which are able to grow and survive when placed in media containing appropriate nutrients and/or growth factors.

The term “cell line” as used herein includes reference to a culture of (for example) eukaryotic cells that can be propagated repeatedly. The eukaryotic cells of the cell line may be selected from any cell as defined herein.

The terms “host cell,” “host cell line” and “host cell culture” are used interchangeably and refer to cells and their progeny into which exogenous nucleic acid can be subsequently introduced to create recombinant cells. These host cells may also have been modified (i.e., engineered) to alter or delete the expression of certain endogenous host cell products (e.g., endogenous virus-like particles or endogenous host cell proteins). Host cells include “transformants” and “transformed cells,” which include the primary transformed cell and progeny derived therefrom without regard to the number of passages. Progeny does not need to be completely identical in nucleic acid content to a parent cell, but can contain mutations. Mutant progeny that have the same function or biological activity as screened or selected for in the originally transformed cell are included herein. The introduction of exogenous nucleic acid (e.g., by transfection) to these host cells would create recombinant cells that are derived from the original “host cell,” “host cell line” or “host cell line”. The terms “host cell,” “host cell line” and “host cell culture” may also refer to such recombinant cells and their progeny. The terms “recombinant cell”, “recombinant cell line” and “recombinant cell culture” are used interchangeably and refer to cells and their progeny into which exogenous nucleic acid has been introduced to enable the expression of recombinant product of interest. The recombinant product expressed by such cells may be a recombinant protein, a recombinant viral particle, or a recombinant viral vector. The term “mammalian host cell” or “mammalian cell” refers to cell lines derived from mammals that are capable of growth and survival when placed in either monolayer culture or in suspension culture in a medium containing the appropriate nutrients and growth factors. The necessary growth factors for a particular cell line are readily determined empirically without undue experimentation, as described for example in Mammalian Cell Culture (Mather, J. P. ed., Plenum Press, N.Y. 1984), and Barnes and Sato, (1980) Cell, 22:649. Typically, the cells are capable of expressing and secreting large quantities of a particular protein of interest into the culture medium. Examples of suitable mammalian host cells within the context of the present disclosure can include Chinese hamster ovary cells/-DHFR (CHO, Urlaub and Chasin, Proc. Natl. Acad. Sci. USA, 77:4216 1980); dp 12.CHO cells (EP 307,247 published 15 Mar. 1989); CHO-K1 (ATCC, CCL-61); baby hamster kidney cells (BHK, ATCC CCL 10); mouse sertoli cells (TM4, Mather, Biol. Reprod., 23:243-251 1980); canine kidney cells (MDCK, ATCC CCL 34); HEK 293 cells; buffalo rat liver cells (BRL 3A, ATCC CRL 1442); mouse mammary tumor (MMT 060562, ATCC CCL51). Exemplary mammalian cells include Chinese hamster ovary cells (CHO). The cells may comprise a polynucleotide that encodes a polypeptide.

The term “biologic” or “biologics” as used herein refers to molecules that are produced by cells, such as such as proteins, glycans, or lipids. The biologics may be biologically active molecules. A biologic may be a recombinant polypeptide, for example a recombinant polypeptide selected from a fusion protein, an antibody, an antigen, an enzyme, and a vaccine. Biologics may be excreted by the cells into the cell culture fluid, which may facilitate isolation of the biologics.

The term “expression” or “expresses” are used herein to refer to transcription and translation occurring within a host cell. The level of expression of a product gene in a host cell can be determined on the basis of either the amount of corresponding mRNA that is present in the cell or the amount of the protein encoded by the product gene that is produced by the cell. For example, mRNA transcribed from a product gene is desirably quantitated by northern hybridization. Sambrook et al., Molecular Cloning: A Laboratory Manual, pp. 7.3-7.57 (Cold Spring Harbor Laboratory Press, 1989). Protein encoded by a product gene can be quantitated either by assaying for the biological activity of the protein or by employing assays that are independent of such activity, such as western blotting or radioimmunoassay using antibodies that are capable of reacting with the protein. Sambrook et al., Molecular Cloning: A Laboratory Manual, pp. 18.1-18.88 (Cold Spring Harbor Laboratory Press, 1989). When reference is made to reduction and/or elimination of the expression of one or more endogenous products relative to the expression of the endogenous product(s) in an unmodified cell, such reductions and/or eliminations of expression encompass reductions and/or eliminations of the active endogenous product, notwithstanding the presence of mRNA encoding all or a portion of the endogenous product or the presence of endogenous product translated from such mRNA.

As used herein, “polypeptide” refers generally to peptides and proteins having more than about ten amino acids. The polypeptides can be homologous to the host cell, or preferably, can be exogenous, meaning that they are heterologous, i.e., foreign, to the host cell being utilized, such as a human protein produced by a Chinese hamster ovary cell, or a yeast polypeptide produced by a mammalian cell. In certain embodiments, mammalian polypeptides (polypeptides that were originally derived from a mammalian organism) are used, more preferably those which are directly secreted into the medium.

The term “protein” is meant to refer to a sequence of amino acids for which the chain length is sufficient to produce the higher levels of tertiary and/or quaternary structure. This is to distinguish from “peptides” or other small molecular weight drugs that do not have such structure. Typically, the protein herein will have a molecular weight of at least about 15-20 kD, preferably at least about 20 kD. Examples of proteins encompassed within the definition herein include host cell proteins as well as all mammalian proteins, in particular, therapeutic and diagnostic proteins, such as therapeutic and diagnostic antibodies, and, in general proteins that contain one or more disulfide bonds, including multi-chain polypeptides comprising one or more inter-and/or intrachain disulfide bonds.

The term “glycoprotein” refers to a protein which contains an oligosaccharide chain covalently attached to amino acid side-chains. The oligosaccharide(s) may be attached to the protein in a co-translational or post-translational modification, during a process known as glycosylation. Exemplary glycoproteins include antibodies, which typically have an N-linked oligosaccharide on each heavy chain.

The term “antibody” is used herein in the broadest sense and encompasses various antibody structures including, but not limited to, monoclonal antibodies, polyclonal antibodies, monospecific antibodies (e.g., antibodies consisting of a single heavy chain sequence and a single light chain sequence, including multimers of such pairings), multispecific antibodies (e.g., bispecific antibodies) and antibody fragments so long as they exhibit the desired antigen-binding activity. A therapeutic antibody is an antibody that may be used in the treatment of a disease.

An “antibody fragment,” “antigen-binding portion” of an antibody (or simply “antibody portion”) or “antigen-binding fragment” of an antibody, as used herein, refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)2; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The term “chimeric” antibody refers to an antibody in which a portion of the heavy and/or light chain is derived from a particular source or species, while the remainder of the heavy and/or light chain is derived from a different source or species.

The term “titer” as used herein refers to the total amount of recombinantly expressed antibody produced by a cell culture divided by a given amount of medium volume. Titer is typically expressed in units of milligrams of antibody per milliliter or liter of medium (mg/ml or mg/L). In certain embodiments, titer is expressed in grams of antibody per liter of medium (g/L). Titer can be expressed or assessed in terms of a relative measurement, such as a percentage increase in titer as compared obtaining the protein product under different culture conditions.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the presently disclosed subject matter can be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human complementarity determining regions (CDRs) and amino acid residues from human framework regions (FRs). In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally can comprise at least a portion of an antibody constant region derived from a human antibody. A “humanized form” of an antibody, e.g., a non-human antibody, refers to an antibody that has undergone humanization.

The term “enzyme” as used herein refers to a protein or polypeptide (such as a recombinant polypeptide) that acts as a biological catalyst.

As used herein, the terms “recombinant protein” and “recombinant polypeptide” refer generally to peptides and proteins, including antibodies, that are encoded by a nucleic acid that is “heterologous,” i.e., foreign to the host cell being utilized, such as a nucleic acid encoding a human antibody that is introduced into a non-human host cell.

### Detailed Description of an Example

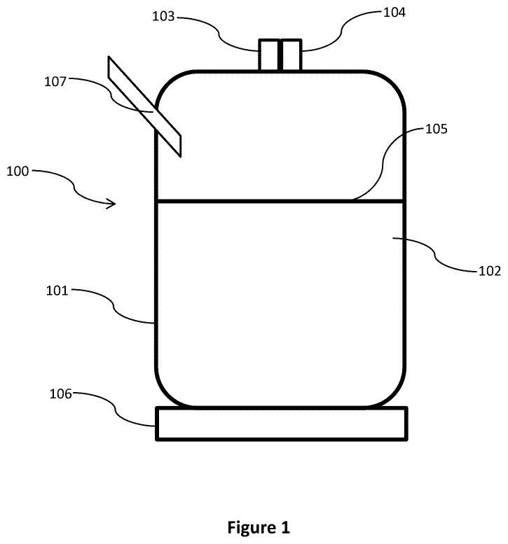

In FIG. 1, an apparatus 100 is shown. The apparatus 100 has a vessel 101. The vessel is capable of holding a liquid 102 within an interior of the vessel 101. The vessel 101 is formed, at least in part, of stainless steel; glass; ceramic; and/or a plastic film or any other suitable materials as appropriate to manufacturing process. The liquid 102 may be added or removed from the vessel 101 at will (for example from one or more inlet or outlet ports (not illustrated), and the quantity of liquid 102 can be controlled or predetermined. Here quantity may be specified in any suitable units, such as, but not limited to mass or volume. The vessel may be sealed or gated (by way of a valve) from the external environment (i.e. the vessel 101 comprises a cavity) or the vessel may be open to the environment, provided the vessel is still configured to hold liquid. The vessel may also be configured to provide sparging (not shown) of held liquid 102 with air, oxygen, or any other suitable gas and/or the vessel may comprise an agitator (not shown) for mixing any held liquid 102.

The apparatus 100 is further equipped with phase surface level determining means. The phase surface level determining means, comprising an emitter 103 and a receiver 104, may be attached on the vessel 101, or be separate from the vessel 101. Where the phase surface level determining means is separate from the vessel 101, the phase surface level determining means may still be arranged in contact with the vessel (if not attached), or may be spaced apart from the vessel, provided the spacing does not prevent the phase surface level determining means performing its intended function. The phase surface level determining means provides the function of detecting the presence and location of the transition between two phases as matter, such as liquid-gas or foam-gas in the present application. In the present application the phase surface level determining means comprises an emitter 103 and a receiver 104.

The emitter 103 is configured to emit a signal towards the interior of the vessel 101, such that the location of the phase surface 105 lies along the path the signal travels. The signal emitted may be as per any signal discussed in this application, such as signal radio waves in the range 26 GHz-170 GHz. As the phase surface 105 lies along the path of travel of the signal, the phase surface may act as a reflector for the signal. The signal, when it encounters the phase surface, is reflected. The whole of the emitted signal may not be reflected. A partial signal may be reflected by the phase surface. The amount of signal reflected may vary depending on the angle of incidence of the signal with the phase surface, or the nature of the phase surface (i.e. particular phases of matter and physical/chemical properties of the materials forming the phase surface at any given time).

The receiver 104 is configured to detect the signal, or the portion of the signal that is reflected back towards the receiver 104. It is noted that the portion of the signal that is reflected back towards the receiver may not be the totality of the reflected signal. The phase surface 105 may scatter a portion of the reflected signal, such that the path of parts of the reflected signal are not co-incident with the receiver 104. The receiver may be arranged adjacent with the emitter, the receiver and emitter may form part of a single unit. Alternatively, the emitter and receiver may be separate units arranged adjacent one another. Further alternatively, the emitter and receiver may be arranged in separate positions, spaced apart from one another provided the receiver is arranged such that the receiver is capable of receiving a signal emitted from the emitter following reflection from the phase surface.

The vessel 101 comprises a wall configured to hold the liquid 102. As the emitter 103 and receiver 104 are arranged external to the vessel, the vessel wall should be formed of a signal transparent material, at least proximate to where the emitter 103 and receiver 104 are situated, in order to allow the signal to pass through.

A liquid level determining means 106 is also provided. The liquid level determining means may be any as described in this application. As previously discussed the level of the liquid may be determined directly or indirectly. Direct methods include ranging apparatus and/or guided wave radar. Indirect methods include load cell or a scales. The vessel 101 in the figures is shown arranged on a scales as the liquid level determining means 106.

A processor (not shown) is provided in communication with the liquid level determining means 106, the emitter 103, and the receiver 104 and arranged to receive signals from those elements.

The apparatus 100 is further provided with an injector 107 which is arranged to release a pre-determined dosage of antifoaming agent into the vessel.

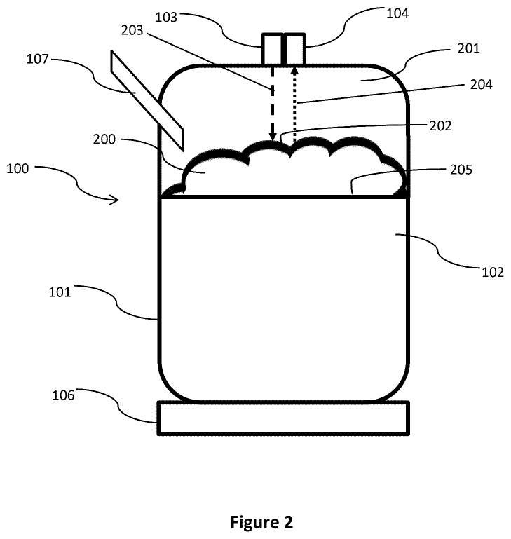

With reference to FIG. 2, there is shown the apparatus 100 of FIG. 1, and having the same features and properties as previously described. The vessel 101 comprises a liquid 102 and a process is underway in the vessel. As a result, a foam 200 has formed on top of the liquid 102, typically between the top of the liquid and a head space 201 of the vessel 101.

The head space 201 is a working area of the vessel 101 which is not presently occupied by the liquid and/or foam. In other words, the head space 201 is the space in the vessel which is not occupied by the liquid or a foam. The head space 201 will change in dimensions depending on changes in volume of the liquid and/or foam (e.g. by addition or expansion of the liquid volume). The head space 201 is typically occupied by a gas.

The foam 200 comprises a liquid contact surface 205 between the liquid 102 and the foam 200, and a phase surface 202 between the foam 200 and the head space 201.

The emitter 103 is shown emitting a signal 203 towards the foam-head space phase surface 202. The emitted signal 203 is reflected on the phase surface, and at least a portion of the reflected signal 204 is detected by the receiver 104. A time of flight of the signal is calculated by the time it takes from the emission of the emitted signal 203 to be detected as a reflected signal 204 at the receiver 104. The distance of the foam-head space surface relative to the receiver 104 may therefore be determined with short durations indicating that the foam-head space phase surface 202 is relatively closer to the receiver 204 than detections having longer durations.

The liquid level determining means 106 may, at the same time as the detection of the signal, provide a value for the liquid level in the vessel, absent foam, which may be used to determine the volume of the vessel that would be occupied by the liquid without a foam, with the remainder of the vessel interior being available head space 201.

The available head space 201 may then be compared to the distance of the foam-head space surface relative to the receiver 104 and thereby determine the thickness of the foam. If the thickness of the foam detected is outside acceptable values, the processor may instruct the dosage of an antifoaming additive to be altered. The dosage may be determined as previously described.

Antifoaming additive may be provided per the calculated dosage via the injector 107 into the vessel 101 to suppress the foam.

Monitoring of the thickness of the foam via detection of the foam-head space phase surface and liquid level determining means may then continue, repeated continuously, until the foam forming process within the vessel ends. In this way the thickness of the foam within the vessel may be automatically controlled without external input. The method is not reliant upon viewing the contents of the vessel and is capable of operating with variations in the liquid quantity.

The acceptable thickness of the foam may be defined as a threshold, which once surpassed, triggers the dosage of the antifoaming agent to be altered. In an example, the alteration of the dosage comprises no addition of the antifoaming agent when the measured thickness of the foam is below the threshold, and addition of an aliquot (e.g. a constant quantity) of the antifoaming agent when the measured thickness of the foam is above the threshold.

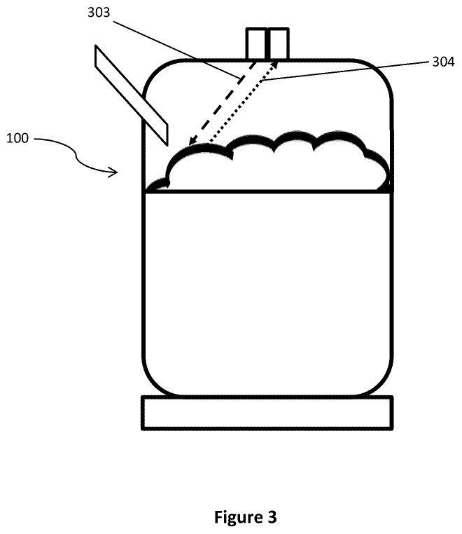

With reference to FIG. 3, there is shown the apparatus 100 of FIGS. 1 and 2, having the same features and properties as previously described. Like reference numerals have been omitted so as to not unnecessarily obscure the figure. The emitter and receiver may be arranged to scan or otherwise take readings across the surface of the foam-head space phase surface. This can be across the whole area of the foam-head space phase surface or another predetermined area.

The emitted signal 303 and the detected signal 304 are shown interrogating the foam-head space phase surface at a location distant from the emitter 103 and the receiver 104. It may be that the acceptable thickness of the foam within the vessel may be non-uniform, with areas of the head space within the vessel tolerating a larger amount of foam compared to other areas (e.g. those areas near filters for example).

Interrogating multiple locations across the foam-head space phase surface also allows for the identification of areas of increased foam thickness relative to other areas as foam formation within the vessel may be non-uniform.

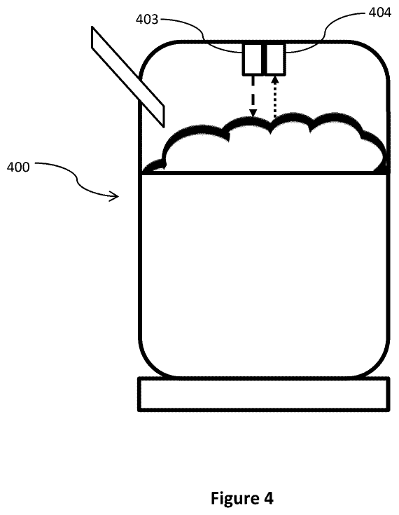

FIG. 4 depicts apparatus 400 similar to that of FIGS. 1 and 2, having the same features and properties as previously described. Like reference numerals have been omitted so as to not unnecessarily obscure the figure. The apparatus 400 differs from the apparatus of earlier figures in that the emitter 403 and the receiver 404 are arranged within the head space of the vessel rather than outside the vessel as shown in FIGS. 1, 2, and 3. Other arrangements are possible as described elsewhere. As the emitter 403 and the receiver 404 are arranged within the head space, the vessel need not be formed of a signal transparent material, and may instead be formed of a signal opaque material.

### Additional Embodiments

Further embodiments of the invention are described in the below numbered clauses:
Clause 1. A method of monitoring and controlling the foam within a vessel comprising a liquid, the method comprising:
a) measuring the thickness of the foam within a vessel comprising a liquid; and
b) controlling the dosage of an antifoaming additive to be added to the liquid based on the measured thickness of the foam;
   wherein the thickness of the foam in step a is determined based on the difference between the level of a phase surface and the level of the liquid; and
   wherein the level of the phase surface is determined by a signal emitted towards and reflected from the phase surface.
Clause 2. The method of clause 1, wherein the phase surface is the surface between foam and headspace gas in the vessel.
Clause 3. The method of clause 1, wherein the level of the phase surface is determined based on a time elapsed between emission of the signal and detection of the reflected signal.
Clause 4. The method of any of clause 1 to clause 3, wherein the signal comprises any of: electromagnetic radio waves; or radio waves.
Clause 5. The method of any previous clause, wherein the signal comprises radio waves in the range 26 GHz-170 GHz.
Clause 6. The method of any previous clause, wherein the signal is emitted towards and reflected from varying locations of the phase surface.
Clause 7. The method of any previous clause, wherein the level of the liquid is determined by volume determination and/or determination of a dimension associated with the liquid.
Clause 8. The method of clause 7, wherein the level of the liquid is determined by determining the volume of the liquid.
Clause 9. The method of clause 8, wherein the volume determination is based on the mass of the liquid.
Clause 10. The method of any previous clause, wherein the level of the phase surface and the level of the liquid are each determined substantially simultaneously.
Clause 11. The method of any previous clause, wherein the dosage of the antifoaming additive is controlled by the comparison of the measured thickness of the foam to a threshold thickness of the foam.
Clause 12. The method of any previous clause, wherein the dosage of the antifoaming additive is, at least in part, determined by the deviation of the determined level from the threshold level of clause 11.
Clause 13. The method of any previous clause, wherein the threshold level of clause 9 is, at least in part, determined based upon an available head space in the vessel.
Clause 14. The method of any previous clause, wherein the dosage of the antifoaming additive is controlled by adjusting the interval of adding the antifoaming additive and/or the amount of the antifoaming additive added in one instance.
Clause 15. The method of any previous clause, wherein the vessel is a bioreactor of a volume in the range of from about 1 L to about 50,000 L.
Clause 16. The method of any previous clause, wherein the liquid is a cell culture fluid comprising cell culture medium and cells.
Clause 17. The method of any previous clause, wherein the method is performed for a duration of a batch culture, a fed-batch culture, or a perfusion culture.
Clause 18. The method of any previous clause, wherein the method is repeated multiple times for the duration of a culture.
Clause 19. The method of clause 18, wherein the repetition is prompted by addition or subtraction of an amount of liquid from the vessel and/or the elapsing of a period of time.
Clause 20. The method of clause 16, wherein the method is repeated substantially continuously for the duration of the culture.
Clause 21. A method of producing a biologic from cells, comprising:
a) culturing the cells in a cell culture fluid in a bioreactor;
b) measuring the thickness of a foam within the bioreactor comprising the cell culture fluid;
c) controlling the dosage of an antifoaming additive to be added to the cell culture fluid based on the measured thickness of the foam; and
d) obtaining the biologic produced by the cultured cells;
   wherein the thickness of the foam in step b is determined based on the difference between the level of a phase surface and the level of the cell culture fluid; and
   wherein the phase surface is determined by a signal emitted towards and reflected from the phase surface.

Claims 22. The method of clause 21, wherein the phase surface is the surface between foam and headspace gas in the vessel.
Clause 23. The method of clause 21, wherein the level of the phase surface is determined based on a time elapsed between emission of the signal and detection of the reflected signal. Clause 24. The method of any of clauses 21 to 23, wherein the signal comprises any of: electromagnetic radio waves; or radio waves.
Clause 25. The method of any one of clauses 21 to 24, wherein the signal comprises radio waves in the range 26 GHz-170 GHz, and optionally wherein the signal comprises 80 GHz radio waves.
Clause 26. The method of any one of clauses 21 to 25, wherein the signal is emitted towards and reflected from varying locations of the phase surface.
Clause 27. The method of any one of clauses 21 to 26, wherein the level of the cell culture fluid is determined by volume determination and/or determination of a dimension associated with the cell culture fluid.
Clause 28. The method of clause 27, wherein the level of the cell culture fluid is determined by volume determination associated with the cell culture fluid, and wherein the volume determination is based on a mass of the cell culture fluid.
Clause 29. The method of any one of clauses 21 to 28, wherein the level of the phase surface and the level of the cell culture fluid are each determined substantially simultaneously.
Clause 30. The method of any one of clauses 21 to 29, wherein the dosage of an antifoaming additive is controlled by the comparison of the measured thickness of the foam to a threshold thickness of the foam.
Clause 31. The method of any one of clauses 21 to 30, wherein the dosage of the antifoaming additive is, at least in part, determined by the deviation of the determined level from the threshold level of clause 27.
Clause 32. The method of any one of clauses 21 to 31, wherein the threshold level of clause 27 is, at least in part, determined based upon an available head space in the vessel.
Clause 33. The method of any one of clauses 21 to 32, wherein the dosage of the antifoaming additive is controlled by adjusting the interval of adding the antifoaming additive and/or the amount of the antifoaming additive added in one instance.
Clause 34. The method of any one of clauses 21 to 33, wherein the bioreactor has a volume in the range of from about 1 L to about 50,000 L.
Clause 35. The method of any one of clauses 21 to 34, wherein the cell culture fluid comprises cell culture medium and cells.
Clause 36. The method of any one of clauses 21 to 35, wherein the steps b) and c) are performed for a duration of a batch culture, a fed-batch culture, or a perfusion culture.
Clause 37. The method of any one of clauses 21 to 36, wherein the steps b) and c) are repeated multiple times for the duration of the culture.
Clause 38. The method of clause 37, wherein the repetition is prompted by addition or subtraction of an amount of cell culture fluid from the vessel and/or the elapsing of a period of time.
Clause 39. The method of any one of clauses 36 to 38, wherein the steps b) and c) are repeated substantially continuously for the duration of the culture.
Clause 40. The method of any one of clauses 36 to 39, wherein step d) is only performed at about the end of the duration of the culture.
Clause 41 The method of any one of clauses 36 to 39 wherein step d) comprises isolating the biologic from the cell culture fluid.
Clause 42. The method of any one of clauses 21 to 41, wherein the biologic is at least one recombinant polypeptide and the cells are eukaryotic cells comprising a polynucleotide encoding the recombinant polypeptide.
Clause 43. The method of clause 42, wherein the at least one recombinant polypeptide is selected from a fusion protein, an antibody, an antigen, an enzyme, or a vaccine.
Clause 44. Apparatus for controlling the thickness of the foam within a vessel comprising a liquid, the apparatus comprising:
a vessel configured to comprise a liquid;
   phase surface level determining means, configured to determine the level of the phase surface, which comprises;
   an emitter, configured to emit a signal towards a phase surface;
   a receiver, configured to detect a reflected signal from the phase surface;
   liquid level determining means, configured to determine the level of the liquid in the vessel; and
a processor, configured to;
   determine the thickness of a foam based on the difference between the level of the phase surface and the level of the liquid; and
   instruct the dosage of an antifoaming additive to be added to the liquid based on the determined thickness of the foam.
Clause 45. The apparatus of clause 44, wherein the phase surface level determining means determines the level of the phase surface based on a time elapsed between emission of the signal toward the phase surface and detection of the reflected signal.
Clause 46. The apparatus of clauses 44 or 45, wherein the emitter and/or receiver are arranged substantially perpendicular to the phase surface of the liquid in the vessel.
Clause 47. The apparatus of any one of clauses 44 to 46, wherein the emitter and/or receiver are arranged above the top of the vessel.
Clause 48. The apparatus of any one of clauses 44 to 47, wherein the emitter and/or receiver are arranged on an outside of the vessel to respectively emit or detect signal to an inside of the vessel.
Clause 49. The apparatus of any one of clauses 44 to 48, wherein the emitter and/or receiver are arranged on an inside of the vessel to respectively emit or detect signal within the vessel. Clause 50. The apparatus of any one of clauses 44 to 49, wherein the emitter is configured to emit any of: electromagnetic radio waves; or radio waves.
Clause 51. The apparatus of clause 50, wherein the emitter is configured to emit radio waves in the range 26 GHz-170 GHz.
Clause 52. The apparatus of any one of clauses 44 to 51, wherein the liquid level determining means is selected from a load cell, a scales, a ranging apparatus, and a guided wave radar.
Clause 53. The apparatus of any one of clauses 44 to 52, wherein the vessel is a bioreactor of a volume in the range of from about 1 L to about 50,000 L.
Clause 54. The apparatus of any one of clauses 44 to 53, wherein the vessel is formed, at least in part, of a signal-transparent material, and wherein the emitter and/or receiver is arranged to respectively emit or detect a signal through the signal-transparent material.
Clause 55. The apparatus of any one of clauses 44 to 54, wherein a top of the vessel is formed of a signal-transparent material.
Clause 56. The apparatus of any one of clauses 54 to 55, wherein the vessel is formed, at least in part, of a radio-opaque material.
Clause 57. The apparatus of any one of clauses 54 to 56, wherein the vessel is formed, at least in part, of stainless steel; glass; ceramic; and/or a plastic film.
Clause 58. The apparatus of any one of clauses 54 to 57, wherein the vessel comprises at least a partial stainless steel shell and a plastic film liner.
Clause 59. The apparatus of any one of clauses 54 to 58, wherein the processor controls the dosage of an antifoaming additive by comparing the determined thickness of the foam to a threshold thickness of the foam.
Clause 60. The apparatus of clause 59, wherein the processor controls the dosage of an antifoaming additive by determining the deviation of the determined level from the threshold level.
Clause 61. The apparatus of any one of clauses 44 to 60, wherein the processor is configured to determine the thickness of the foam and instruct the dosage of an antifoaming additive repeatedly and/or continuously during a cell culture duration.
Clause 62. The apparatus of any one of clauses 44 to 61, wherein the apparatus further comprises an antifoaming additive injector configured to automatically inject the antifoaming additive into the vessel when the processor instructs the addition of the antifoaming additive.
Clause 63. Use of an apparatus of any of clauses 44 to 62 for controlling the thickness of the foam in a vessel, optionally wherein the vessel is a bioreactor.
Clause 64. Use of an apparatus of any of clauses 44 to 62 for culturing a cell line.
Clause 65. The use of the apparatus of clause 64, wherein the cell line is engineered to produce a recombinant polypeptide;
optionally wherein the recombinant polypeptide is a fusion protein, an antibody, an antigen, an enzyme, or a vaccine.
Clause 66. Use of an apparatus of any of clauses 44 to 62 for the production of a biologic, wherein the vessel comprises a cell culture fluid that comprises a cell line engineered to produce said biologic.
Clause 67. The use of an apparatus as disclosed in clause 66, wherein the biologic is at least one recombinant polypeptide and the cell line is a eukaryotic cell line comprising a polynucleotide encoding the recombinant polypeptide;
optionally wherein the recombinant polypeptide is a fusion protein, an antibody, an antigen, an enzyme, or a vaccine.
Clause 68. The use of clause 66 or clause 67, further comprising isolating the biologic.

Any antifoaming additive or agent suitable for mammalian or prokaryotic cell culture may be used for the methods and other embodiments disclosed herein. In any of the embodiments herein, the antifoaming additive or agent is a silicone based antifoam agent, comprising for example, one or more of a simethicone; a polydimethyl siloxane; and/or a silicon dioxide. IN certain embodiments, the antifoam additive or agent is LIVEO™ (DuPont™).

### EXAMPLES

### Example 1: Continuous Monitoring of the Foam Within a Vessel

An apparatus comprising a bioreactor was provided with a radar (80 GHz) positioned proximate to the top of the bioreactor and a load cell. The radar comprised an emitter and a receiver, was positioned proximate to the top of the bioreactor and was configured to emit and receive a signal to enable determination of the level of a phase surface in the bioreactor. The load cell was used to measure the mass of the cell culture in a bioreactor, which was converted to the level of the liquid, using a previously determined calibration, based on the volume and geometry of the bioreactor. The bioreactor further comprised inlets for separate addition of cell culture feed and antifoam additive. The bioreactor also comprised means for gas sparging. The bioreactor was in communication with a processor. The processor was configured to determine the liquid level from the output of the load cell, determine the thickness of the foam from the difference between the level of the phase surface and the level of the liquid, and automatically add antifoam additive based on a pre-planned schedule.

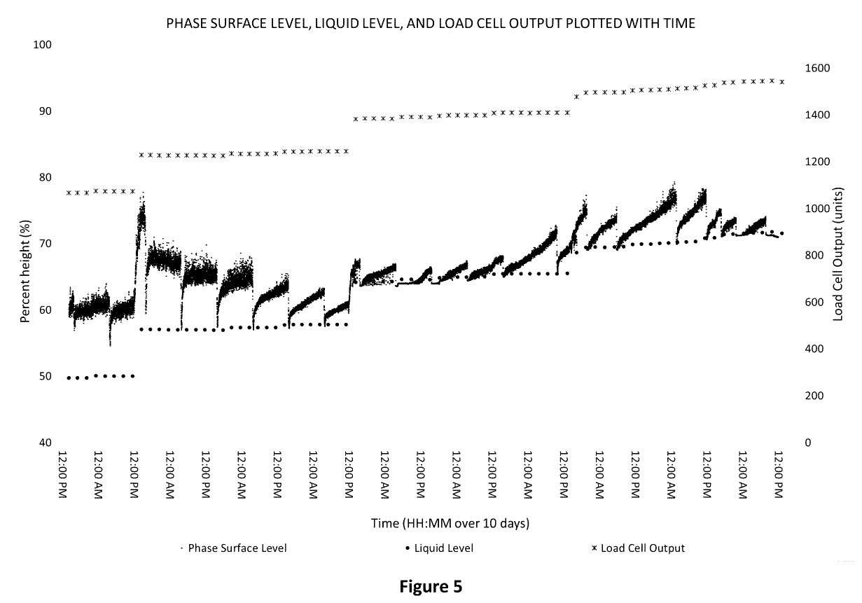

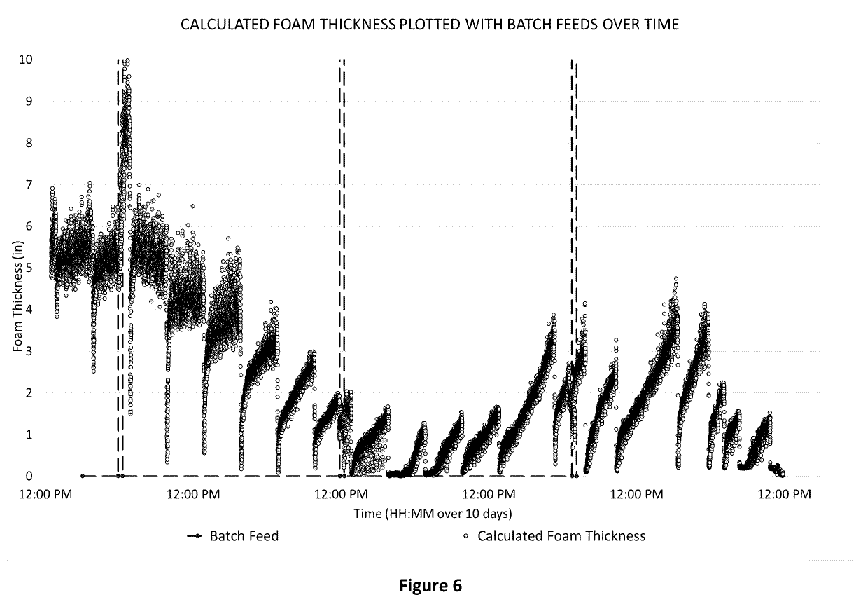

An experiment was performed to confirm the ability of the apparatus to continuously measure the thickness of the foam in the bioreactor over an extended period of time. Results are provided in FIGS. 5 and 6.

FIG. 5 is a plot of the phase surface level, the load cell output, and the converted liquid level from the load cell output over time in an implementation of the disclosure. The x-axis shows the passage of time over the course of 10 days. The output of the load cell is shown with changes during a manufacturing process progresses. The units of the load cell output can be any suitable as described herein, provided the output of the load cell can be used to determine the liquid level, absent foam. The changes in load cell output could be, by way of non-limitative example, changes in the liquid volume either through addition/subtraction of liquid and/or growth of a product in the vessel in a bioprocess.

The liquid level, expressed as percent height of the vessel, is calculated from the load cell output in FIG. 5. As can be seen (and as would be expected), the liquid level closely follows the load cell output due to its cylindrical shape of the vessel in this example. Using a percentage as a unit of the vessel for levels of liquid and phase surface provides an advantage to make the calculation in a processor for the foam thickness more easily transferrable to other vessels.

In FIG. 5, the phase surface level measured by the radar is expressed as percent height of the vessel. The liquid level converted from a load cell is also expressed as percent height of the vessel. Subtracting the percent of the liquid level occupied in the vessel from the percent of the phase surface level provides the percent of the foam thickness in terms of vessel height, which is then converted to the actual foam thickness in inch in this case at any given moment as shown in FIG. 6. As can be seen, the foam thickness was determined using the techniques as hereinbefore described and plotted. When the foam was generated in the bioreactor vessel, the phase surface level is greater than the liquid level, and shows the foam thickness in inch is greater than 0. When there was no foam in the bioreactor, the foam thickness is zero.

FIG. 6 shows thickness of the foam in circled dots determined by the measurements of the phase surface level and the liquid level displayed in FIG. 5. The x-axis shows the passage of time over the course of 10 days. Periodic batch feeds (dashed line) were made in the process, three times in this process. Foam was produced as the manufacturing process proceeded (e.g. from gas production and/or sparging). An antifoaming additive was added to the liquid over the 10 days by predetermined schedule. As shown in FIG. 6, the apparatus continuously measured the thickness of the foam in the bioreactor over an extended period of time.

### Example 2: Control of the Dosage of an Antifoaming Additive Based on the Foam Thickness

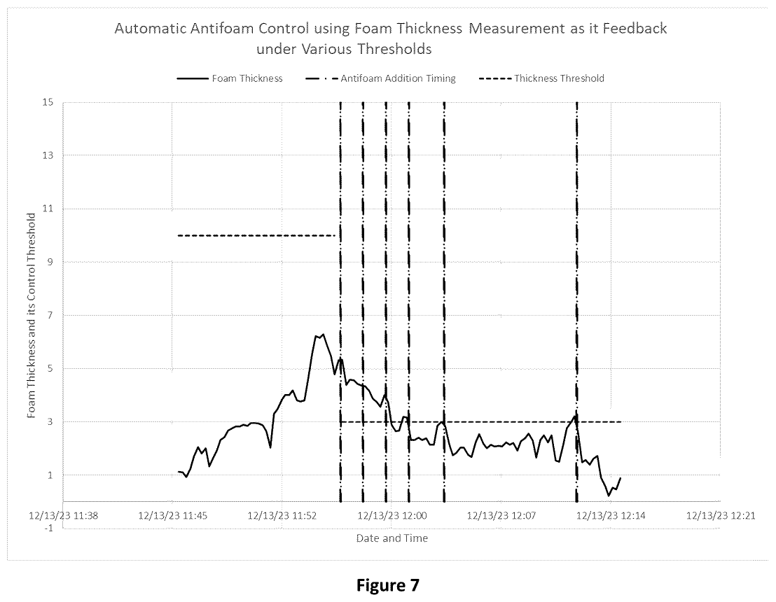

FIG. 7 shows automatic antifoam control using the foam thickness measurement compared to a threshold thickness. The foam thickness in the vessel (solid line) was measured in the same manner as Example 1 and compared with a threshold thickness. The threshold thickness (dotted line on horizontal axis) was initially set to 10 inches and then switched to 3 inches. The dosage was set to add 20 g antifoam additive after 45 seconds from the end of the previous antifoam additive addition if the foam thickness was over the threshold thickness. In this figure, during the period when the threshold thickness was set to 10 inches, there was no antifoam additive addition because the foam thickness was always under the threshold value. During the period when the threshold thickness was set to 3 inches, the first four doses were made following the dosage plan because the foam thickness was over the threshold thickness. In the 5^{th} and 6^{th} doses, however, the interval was adjusted so that there was no antifoaming addition until the foam thickness meet or exceeded the threshold value.

The threshold may be set in more than one way. For example, the threshold may be set at a specified level of the foam in the vessel relative to the level of the liquid at that time. In another example, the threshold could be set at a rate of change of the thickness of the foam, with antifoaming agent added when a rapid increase of the thickness of the foam is detected within the vessel.

FIG. 7 shows the successful automatic management of foam within a vessel using the disclosed methods. This provides important advantages (e.g. for cell culturing processes), as it both ensures that the level of foam does not get too high, and also avoids the addition of more antifoaming additive than is necessary.

### Example 3: Example Bioreactor Cell Culture With Automatic Foam Control

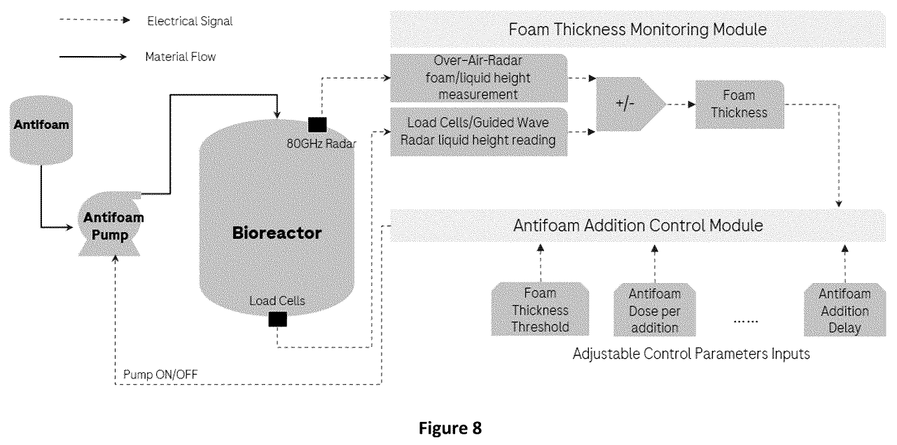

FIG. 8 depicts an example automatic anti-foam control bioreactor cell culture system. A 2000 L bioreactor was loaded with 1200 L CHO cell culture medium and inoculated from a standard cell culture seed train. The bioreactor was equipped with load cells that detect and report on the mass of the cell culture in a bioreactor, which was converted to the level of the liquid based on the volume and geometry of the bioreactor. The load cells can be replaced by using a guide-wave radar for a direct measurement of liquid height. The bioreactor further comprised an 80 GHz radar positioned proximate to the top of the bioreactor. The radar was configured to emit and receive a signal to enable determination of the level of a phase surface in the bioreactor. In this instance an over-air radar was used to measure liquid or foam height, but the system can use a guided wave radar instead or in addition.

During cell culture, which includes sparging the cell culture medium with gases (e.g., oxygen), foam developed and collected on top of the cell culture medium.

The bioreactor was connected to an antifoam addition pump, which added an amount of liquid antifoam agent (e.g., Liveo™ (DuPont™), a silicone based antifoam agent, comprising for example, one or more of a simethicone; a polydimethyl siloxane; and/or a silicon dioxide) into the bioreactor when the foam reaches a predetermined height. The amount of antifoam agent (dose of antifoam) was calculated for a 2000 L bioreactor; smaller or larger bioreactors would use accordingly less or more antifoam agent per dose.

The activity of the antifoam addition pump was controlled by a Foam Thickness Monitoring Module and an Antifoam Addition Control Module, The Foam Thickness Monitoring Module received liquid height and foam height information from the radar to calculate foam thickness. When the foam height reached a threshold height (that is, when the foam layer rose unacceptably above the cell culture medium), the Antifoam Addition Control Module directed the antifoam addition pump to switch on, dispensing a dose of antifoam into the cell culture medium. The Antifoam Addition Control Module allowed the user to set a threshold acceptable foam height (Foam Thickness Threshold), amount of antifoam to add per dose, amount of time to delay addition of antifoam, etc.

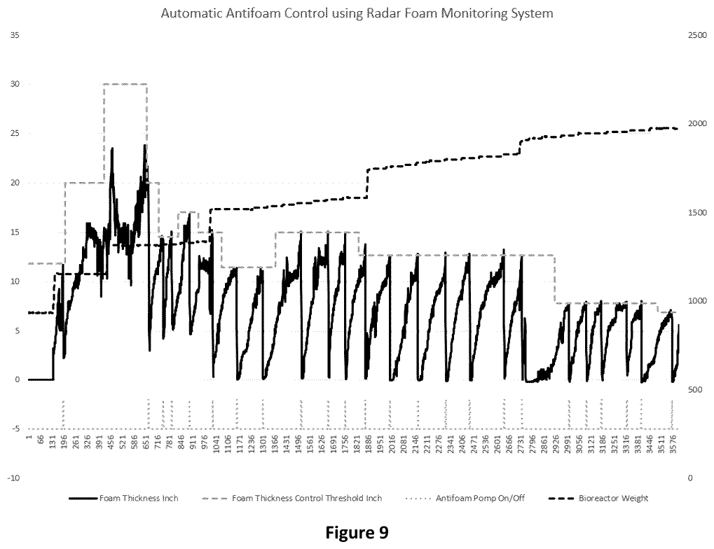

FIG. 9 depicts performance of the antifoam system during cell culture. The X-axis indicates time points of culture after initial inoculation with seed train cell culture. (Note the settling seen at about time point 326 is likely due to foam settling without antifoam addition, and the settling seen at time point 456 is due to a manual foam addition.) The left Y-axis indicates inches of foam in the bioreactor, and the right Y-axis indicates the bioreactor weight in kilograms. The stepped line rising to the right is the weight of the bioreactor upon addition of cell culture medium, seed train cell culture, cell culture medium feedings, and inputs of antifoam. The line at the bottom of the figure indicates times the antifoam pump is on when there is a peak (short vertical line). The stepped line generally descending from left to right is the user-set foam thickness threshold. The jagged line is the actual measured foam thickness during the course of cell culture. This line indicates a rise in foam thickness during cell culture and sparging, with a sharp drop at and just after the point at which the antifoam addition pump is on.

As can be seen in FIG. 9, foam thickness, which was initially allowed to rise to almost 25 inches, when the Antifoam Addition Control Module was set to allow as much as 30 inches of foam), was rapidly responsive to the addition of antifoam when the desired threshold foam thickness was reset. See, e.g., rapid reduction in foam thickness when the foam thickness threshold was reduced at time point minutes from 30 inches to 20 inches, and then foam thickness following the foam thickness threshold changes at time point 716 to 14 inches then at time point 846 to 17 inches, then at time point 920 to 15 inches, as indicated by the sharp drops in foam thickness due to automatic antifoam addition when the foam thickness reaches the set foam thickness threshold. Foam thickness was followed for approximately 3600 minutes cell culture time.

All of the references cited herein are hereby incorporated by reference in their entireties.

1. A method of monitoring and controlling foam within a vessel comprising a liquid, the method comprising:
a) measuring a thickness of the foam within the vessel comprising the liquid; and
b) controlling a dosage of an antifoaming additive to be added to the liquid based on the measured thickness of the foam;
wherein the thickness of the foam in step a) is determined based on the difference between a level of a phase surface and a level of the liquid; and
wherein the level of the phase surface is determined by a signal emitted towards and reflected from the phase surface.

2. The method of claim 1, wherein the phase surface is the surface between the foam and a headspace gas in the vessel.

3. The method of claim 1, wherein the signal comprises radio waves in the range 26 GHz-170 GHz.

4. The method of claim 1, wherein the signal is emitted towards and reflected from varying locations of the phase surface.

5. The method of claim 1, wherein the level of the liquid is determined by: i) volume determination and/or determination of a dimension associated with the liquid; and/or, ii) determination of the volume the liquid.

6. (canceled)

7. The method of claim 5, wherein the volume is determined based on the mass of the liquid.

8. The method of claim 1, wherein the level of the phase surface and the level of the liquid are each determined substantially simultaneously.

9. The method of claim 1, wherein the dosage of the antifoaming additive is:
(i) controlled by the comparison of the measured thickness of the foam to a threshold thickness of the foam; and/or,
(ii) at least, in part, determined by the deviation of the determined level from the threshold level.

10. (canceled)

11. The method of claim 9, wherein the threshold level is, at least in part, determined based upon an available head space in the vessel.

12. The method of claim 1, wherein the dosage of the antifoaming additive is controlled by adjusting the interval of adding the antifoaming additive and/or the amount of the antifoaming additive added in one instance.

13. The method of claim 1, wherein the vessel is a bioreactor of a volume in the range of from about 1 L to about 50,000 L.

14. The method of claim 1, wherein the liquid is a cell culture fluid comprising cell culture medium and cells.

15. The method of claim 14, wherein the method is performed for a duration of a batch culture, a fed-batch culture, or a perfusion culture.

16. The method of claim 15, wherein the method is repeated multiple times for the duration of the culture.

17. The method of claim 16, wherein the repetition is prompted by addition or subtraction of an amount of liquid from the vessel and/or the elapsing of a period of time.

18. The method of claim 12, wherein the method is repeated substantially continuously for the duration of the culture.

19. A method of producing a biologic from cells, comprising:
a) culturing said cells in a cell culture fluid in a bioreactor;
b) measuring the thickness of a foam within the bioreactor comprising the cell culture fluid;
c) controlling the dosage of an antifoaming additive to be added to the cell culture fluid based on the measured thickness of the foam; and
d) obtaining the biologic produced by the cultured cells;
wherein the thickness of the foam in step b is determined based on the difference between the level of a phase surface and the level of the cell culture fluid; and
wherein the phase surface is determined by a signal emitted towards and reflected from the phase surface.

20-41. (canceled)

42. An apparatus for controlling a thickness of a foam within a vessel comprising a liquid, the apparatus comprising:
the vessel configured to comprise the liquid;
a phase surface level determining means, configured to determine the level of the phase surface, which comprises;
an emitter, configured to emit a signal towards the phase surface;
a receiver, configured to detect a reflected signal from the phase surface;
a liquid level determining means, configured to determine the level of the liquid in the vessel; and
a processor, configured to;
(a) determine the thickness of the foam based on a difference between the level of the phase surface and the level of the liquid; and
(b) instruct a dosage of an antifoaming additive to be added to the liquid based on the thickness of the foam determined in step (a).

43-60. (canceled)

61. The method of claim 1, wherein the antifoaming additive is a silicone-based antifoam agent.

62. The method of claim 1, wherein the antifoaming additive comprises one or more of a simethicone; a polydimethyl siloxane; and/or a silicon dioxide.
